(19) United States
(12) Patent Application Publication
(10) Pub. No.: US 2009/0132019 A1
(43) Pub. Date: May 21, 2009

(54) Bifurcate Stent Delivery Catheter

(75) Inventors: Niall Duffy, Ballybrit (IE); Ian Matheson, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/940,515

(22) Filed: Nov. 15, 2007

(51) Int. Cl. A61F 2/84; A61F 2/82

(52) U.S. Cl. 623/1.11; 623/1.35

(57) ABSTRACT

A bifurcate stent delivery catheter is disclosed that includes a proximal shaft and two distal shaft branches having a bifurcated stent mounted thereon, wherein one or more conventional balloon catheters may be advanced within lumens of the bifurcate catheter to deploy the bifurcated stent. Since no balloons are mounted on the bifurcate catheter, the clinician may use any combination of balloon sizes for deploying various portions of the bifurcated stent and thus custom-select appropriate balloon sizes to best treat the bifurcation. Once a conventional balloon catheter is positioned within a distal shaft branch of the bifurcate stent delivery catheter, each of the distal shaft branches of the bifurcate catheter are constructed to split or otherwise expand in a controlled manner upon expansion of the balloon catheter in order to allow the bifurcated stent to be expanded or deployed.

### FIELD OF THE INVENTION

The present invention relates in general to stent delivery systems employed in the treatment of vascular disease. More particularly, the present invention relates to a stent delivery system that has a bifurcated stent attached at the distal end thereof for treatment of vascular disease in arterial bifurcations.

### BACKGROUND OF THE INVENTION

Balloon angioplasty employs balloon tipped catheters to expand the walls of narrowed vessels and to deploy endoluminal prostheses to maintain lumen patency. Although systems and techniques exist that work well in many cases, no technique is applicable to every case. For example, special methods exist for dilating lesions that occur in branched or bifurcated vessels. A bifurcation is an area of the vasculature where a main vessel is bifurcated into two or more branch vessels. It is not uncommon for stenotic lesions to form in such bifurcations. The stenotic lesions can affect only one of the vessels, i.e., either of the branch vessels or the main vessel, two of the vessels, or all three vessels.

Methods to treat bifurcated vessels seek to prevent the collapse or obstruction of the main and/or branch vessel(s) during the dilation of the vessel to be treated. Such methods include techniques for using double guidewires and sequential percutaneous transluminal coronary angioplasty (PTCA) with stenting or the “kissing balloon” and “kissing stent” technique, which provide side branch protection. In addition, in order to effectively treat stenoses at a bifurcation, attempts to simultaneously dilate both branches of the bifurcated vessel have been pursued. These attempts include deploying more than one balloon, more than one prosthesis/stent, a bifurcated or Y-shaped prosthesis/stent, or some combination of the foregoing. Simultaneously deploying multiple and/or bifurcated balloons with or without endoluminal prostheses/stents requires highly accurate placement of the assembly. Specifically, deploying a bifurcate catheter assembly requires positioning a main body of the catheter within the main vessel adjacent the bifurcation, and then positioning the independent distal portions of the catheter assembly into the branch vessels.

Generally, PTCA is a procedure that involves passing a balloon catheter over a guidewire to a stenosis with the aid of a guide catheter. The guidewire extends from a remote incision to the site of the stenosis, and typically across the lesion. The balloon catheter is passed over the guidewire, and ultimately positioned across the lesion. Once the balloon catheter is appropriately positioned across the lesion, e.g., under fluoroscopic guidance, the balloon is inflated to break-up the plaque of the stenosis to thereby increase the vessel cross-section. The balloon is then deflated and withdrawn over the guidewire into the guide catheter to be removed from the body of the patient.

In many cases, a stent or other prosthesis must be implanted to provide permanent support for the vessel. When such a device is to be implanted, a balloon catheter, typically carrying a stent on its balloon, is deployed to the site of the stenosis. The balloon and accompanying stent are positioned at the location of the stenosis, and the balloon is inflated to circumferentially expand and thereby implant the stent. Thereafter, the balloon is deflated and the catheter and the guidewire are withdrawn from the patient. Since the stent is typically mounted on the balloon, the balloon size for deploying the stent is predetermined and cannot be adjusted by the clinician in vivo. However, in some instances, it may be desirable to allow a clinician to select an appropriate balloon size for the body lumen to be treated after the stent has reached the in vivo treatment site.

Implanting a stent at a bifurcation in a body lumen requires additional consideration of appropriate balloon sizes due to the relative sizes of the main vessel and the branch vessels. Some branch vessels can have somewhat smaller diameter lumens than the main vessel from which they branch. In addition, some branch vessels can have lumens with somewhat different diameters from each other. Therefore, balloons of different sizes may be needed for properly deploying a stent in each of the main and branch vessels. It would be desirable to have a stent delivery system that allows a clinician to select an appropriate balloon size for deploying a stent in a particular body lumen, such as an artery, wherein the balloon size may be selected after the stent reaches the treatment site. Especially advantageous in treating a bifurcation, it would be desirable to allow a clinician to custom-select different combinations of balloon sizes for deploying a bifurcated stent in main or branch vessels having different diameter lumens, once the stent has been tracked to the bifurcation to be treated.

### BRIEF SUMMARY OF THE INVENTION

Embodiments of the present invention are directed to a stent delivery system for delivering a stent to a bifurcation. In one embodiment of the present invention, the stent delivery system comprises a proximal shaft having a proximal end and a distal end with a lumen extending there through, a first distal shaft branch having a first branch lumen extending there through with an area of weakness along a length thereof, and a second distal shaft branch having a second branch lumen extending there through with an area of weakness along a length thereof. The first and second distal shaft branches separately extend from the distal end of the proximal shaft such that the first and second branch lumens are in fluid communication with the proximal shaft lumen. Each of the areas of weakness along the first and second distal shaft branches splits open to an expanded configuration when a balloon of a balloon catheter is inflated within its respective lumen.

In another embodiment of the present invention, the stent delivery system comprises a proximal shaft having a proximal end and a distal end with a lumen extending there through, a first distal shaft branch formed of a coiled tube having a first branch lumen extending there through, and a second distal shaft branch formed of a coiled tube having a second branch lumen extending there through. The first and second distal shaft branches separately extend from the distal end of the proximal shaft such that the first and second branch lumens are in fluid communication with the proximal shaft lumen. Each of the first and second distal shaft branches unrolls to an expanded configuration when a balloon of a balloon catheter is inflated within its respective lumen.

In another embodiment of the present invention, the stent delivery system comprises a proximal shaft having a proximal end and a distal end with a lumen extending there through, a first distal shaft branch having a first branch lumen extending there through, and a second distal shaft branch having a second branch lumen extending there through. The first and second distal shaft branches separately extend from the distal end of the proximal shaft such that the first and second branch lumens are in fluid communication with the proximal shaft lumen. At least an integral portion of each of the first and second distal shaft branches is formed of a polymeric material that expands without exceeding its elastic limit to an expanded configuration when a balloon of a balloon catheter is inflated within its respective lumen.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side elevational view of a bifurcate stent delivery catheter having a bifurcated stent mounted thereon in accordance with an embodiment of the present invention.

FIG. 2 is a cross-sectional view of a bifurcate stent delivery catheter in accordance with an embodiment of the present invention taken along line A-A of FIG. 1.

FIG. 3 is a cross-sectional view of a bifurcate stent delivery catheter in accordance with another embodiment of the present invention taken along line A-A of FIG. 1.

FIG. 4 is a cross-sectional view of a bifurcate stent delivery catheter in accordance with an embodiment of the present invention taken along line B-B of FIG. 1.

FIG. 5A is a side elevational view of the distal portion of a bifurcate stent delivery catheter in accordance with an embodiment of the present invention.

FIG. 5B is a side elevational view of the distal portion of a bifurcate stent delivery catheter in accordance with another embodiment of the present invention.

FIG. 5C is a cross-sectional view of the distal portion of a bifurcate stent delivery catheter in accordance with an embodiment of the present invention taken along line C-C of FIG. 5B.

FIG. 5D is a side elevational view of the distal portion of a bifurcate stent delivery catheter in accordance with another embodiment of the present invention.

FIG. 5E is a cross-sectional view of the distal portion of a bifurcate stent delivery catheter in accordance with an embodiment of the present invention taken along line D-D of FIG. 5D.

FIG. 5F is a side elevational view of the distal portion of a bifurcate stent delivery catheter in accordance with another embodiment of the present invention.

FIG. 6 is a perspective view of the distal portion of a bifurcate stent delivery catheter in accordance with another embodiment of the present invention.

FIG. 7 is a side elevational view of a bifurcate stent delivery catheter having a bifurcated stent mounted thereon in accordance with another embodiment of the present invention.

FIG. 8 is an enlarged sectional view of a distal portion of a bifurcate stent delivery catheter with a conventional balloon catheter inserted therein in accordance with an embodiment of the present invention taken along line X-X of FIG. 1.

FIG. 9 is a side elevational view of a bifurcate stent delivery catheter in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

Embodiments of the present invention are directed to a stent delivery catheter having a stent mounted on a distal portion of a catheter shaft. The distal portion of the catheter shaft is designed to split or otherwise expand in a controlled fashion by a conventional balloon catheter inserted within a lumen of the shaft to thereby permit the expansion or deployment of the stent mounted thereon. Thus, embodiments of the present invention do not have a stent mounted directly on a balloon on the distal end of the stent delivery catheter. Instead, a clinician may select an appropriate balloon size, i.e., an appropriate balloon catheter size, to suit the target body lumen for insertion within the stent delivery catheter in accordance with embodiments of the present invention.

Although embodiments of the present invention may be applied to any stent delivery system, the present invention is especially advantageous in a bifurcate stent delivery system having a bifurcated stent at the distal end thereof. Since there are no balloon(s) mounted on the distal end of the bifurcate stent delivery catheter, conventional balloon catheters having different size balloons may be used to expand different portions of the bifurcated stent, as will be explained herein.

An embodiment of the present invention is a bifurcate stent delivery catheter including a proximal shaft and two distal shaft branches having a bifurcated stent mounted thereon, wherein one or more conventional balloon catheters may be advanced within lumens of the bifurcate catheter to deploy the bifurcated stent. Since no balloons are mounted on the bifurcate stent delivery catheter, the clinician may use any combination of balloon sizes for deploying different portions of the bifurcated stent, i.e., the trunk and branches of the stent. As such, the clinician may custom-select appropriate balloon sizes to best treat the bifurcation. Once a conventional balloon catheter is positioned within a distal shaft branch, each of the distal shaft branches of the bifurcate catheter are constructed to split or otherwise expand in a controlled manner in order to allow the bifurcated stent to be expanded or deployed. Conventional balloon catheters that may be used in the present invention includes any type of catheter known in the art, including over-the-wire catheters, rapid-exchange catheters, core wire catheters, and any other appropriate balloon catheters. For example, conventional balloon catheters such as those shown or described in U.S. Pat. Nos. 6,736,827; 6,554,795; 6,500,147; and 5,458,639, which are incorporated by reference herein in their entirety, may be used within the bifurcate stent delivery catheter of the present invention.

Embodiments of the present invention include various ways to construct the distal shaft branches to expand in a controlled manner such that the bifurcated stent may be expanded or deployed by a conventional balloon catheter advanced through the lumen of the distal shaft branches. In one embodiment, the first and second distal shaft branches each have an area of weakness that splits open in response to inflation of a balloon of the conventional balloon catheter. The area of weakness may be a perforation, a slit, a slot, a groove, or a connector such as an adhesive which is breakable by the inflation of a balloon. In another embodiment, the distal shaft branches are constructed of a coiled tube that unrolls in response to inflation of a balloon of the conventional balloon catheter. In yet another embodiment, the distal shaft branches are constructed out of an elastomer material that expands in response to inflation of a balloon of the conventional balloon catheter.

The distal shaft branches typically are constructed out of a more flexible material than the proximal shaft, and typically have a smaller outer diameter than the proximal shaft. Each distal shaft branch includes one lumen there through for accommodating insertion of one conventional balloon catheter.

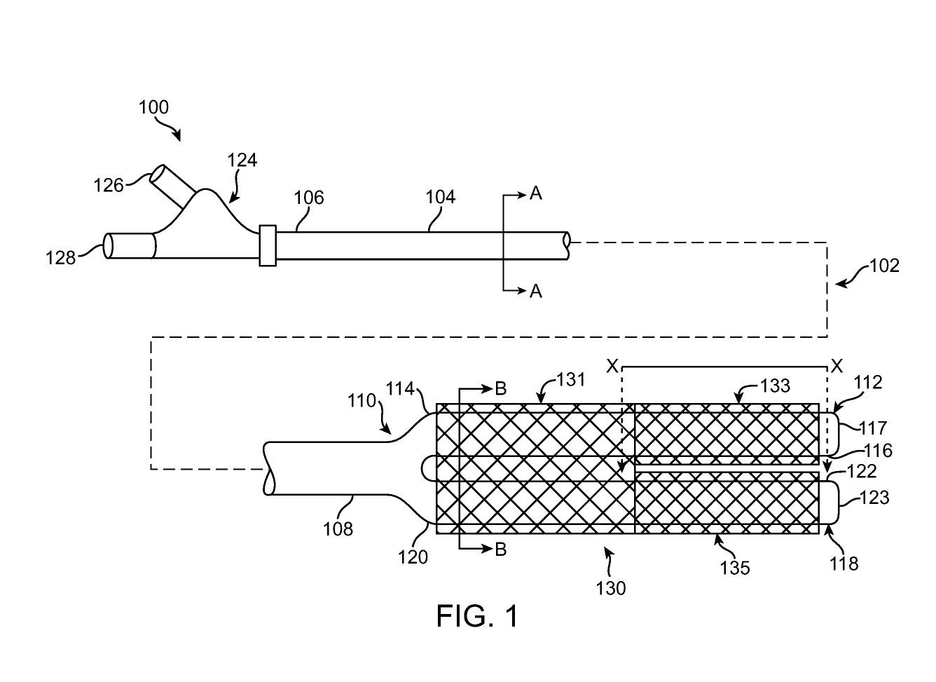

The proximal shaft may be biluminal in order to simultaneously accommodate two conventional balloon catheters and/or guidewires. In such a biluminal embodiment, the lumens of the proximal shaft are each respectively in fluid communication with a lumen of a distal shaft branch. In another embodiment, the proximal shaft has a single lumen in fluid communication with both lumens of first and second distal shaft branches. The single lumen may be of a sufficient size to simultaneously accommodate two conventional balloon catheters inserted there through. Alternatively, the single lumen may be of a sufficient size to accommodate only one conventional balloon catheter. In such an embodiment, a first conventional balloon catheter having a balloon of a first size is inserted in order to expand or deploy a portion of the bifurcated stent mounted on the first distal shaft branch. The first conventional balloon catheter is retracted and removed, and subsequently a second conventional balloon catheter having a balloon of a second size is inserted in order to expand or deploy a portion of the bifurcated stent mounted on the second distal shaft branch. Further details and description of the embodiments of the present invention are provided below with reference to FIGS. 1-9.

FIG. 1 is an illustration of a bifurcate stent delivery system 100 in accordance with an embodiment of the present invention. Bifurcate stent delivery system 100 includes catheter 102 having a bifurcated stent 130 mounted on a distal portion thereof. Catheter 102 includes a proximal shaft 104 having a proximal end 106 attached to a hub 124 and a distal end 108 attached to first and second distal shaft branches 112, 118, respectively, via a junction 110. First and second distal shaft branches 112, 118 separately and independently extend from distal end 108 of proximal shaft 104. First distal shaft branch 112 includes a proximal end 114 and a distal end 116, wherein distal end 116 includes a guidewire exit port 117. Second distal shaft branch 118 includes a proximal end 120 and a distal end 122, wherein distal end 122 includes a guidewire exit port 123. Bifurcated stent 130 is mounted on first and second distal shaft branches 112, 118 at a location between proximal ends 114, 120 and distal ends 116, 122 of first and second distal shaft branches 112, 118, respectively. There are no balloons mounted on either of first or second distal shaft branches 112, 118 under bifurcated stent 130. In general, bifurcated stent 130 consists of three portions or components, a main trunk 131 and first and second branch portions 133, 135, respectively. Bifurcated stent 130 may be any bifurcated stent known in the art such as the stents shown or described in U.S. Pat. No. 6,129,738 to Lashinski et al.; U.S. Pat. No. 7,131,991 to Zarins et al; and U.S. Pat. No. 7,018,400 to Lashinski et al., which are incorporated by reference herein in its entirety. Alternatively, bifurcated stent 130 may comprise two or more separate prostheses to be deployed in the main and/or branch vessels of a bifurcation.

Hub 124 includes a first catheter port 126 and a second catheter port 128 for accommodating insertion of one or more conventional balloon catheters and/or guidewires within first distal shaft branch 112 and second distal shaft branch 118 of catheter 102. First catheter port 126 and second catheter port 128 may be labeled to indicate which distal shaft branch (i.e., first distal shaft branch 112 or second distal shaft branch 118) corresponds to first catheter port 126 and which distal shaft branch (i.e., first distal shaft branch 112 or second distal shaft branch 118) corresponds to second catheter port 128. FIG. 4 is a cross-sectional view of first and second distal shaft branches 112, 118 taken along line B-B of FIG. 1. However, bifurcated stent 130 is not depicted on the cross-sectional view of FIG. 4 for clarity purposes. With reference to both FIG. 4, first and second distal shaft branches 112, 118 each include a lumen 450, 456, respectively, extending there through for accommodating insertion of a guidewire and a conventional balloon catheter. First and second distal shaft branches 112, 118 are each constructed to split or otherwise expand in order to allow bifurcated stent 130 to be expanded or deployed by one or more conventional balloon catheter advanced through respective lumens 450, 456. First distal shaft branch 112 has an outside surface 446 and includes lumen 450 there through. Lumen 450 of first distal shaft branch 112 is defined by inside surface 448. Second distal shaft branch 118 has an outside surface 452 and includes lumen 456 there through. Lumen 456 of second distal shaft branch 118 is defined by inside surface 454.

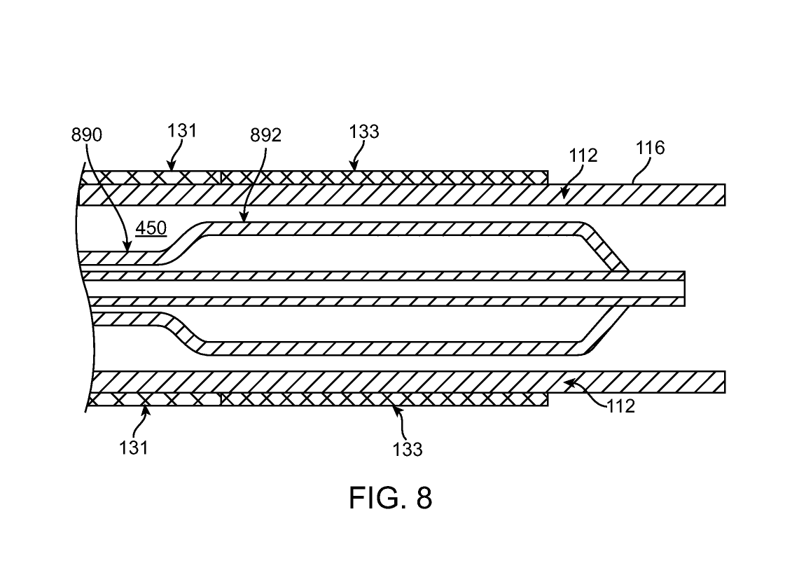

First and second distal shaft branches 112, 118 have distal ends 116, 122 that include guidewire exit ports 117, 123 such that a guidewire may extend completely through lumens 450, 456 of first and second distal shaft branches 112, 118. Two guidewires may be used to properly position first and second distal shaft portions 112, 118 within the branches of the vessel lumen, as described in more detail below. Once bifurcate stent delivery system 100 is in place as desired, conventional balloon catheters are inserted through catheter ports 126, 128 and tracked through the distal shaft branches 112, 118 to a position in which the balloons of the conventional balloon catheters are placed under at least a portion of bifurcated stent 130. To illustrate, FIG. 8 shows an enlarged sectional view of first distal shaft branch 112 taken along line X-X of FIG. 1. First distal shaft branch 112 has a first conventional balloon catheter 890 inserted within lumen 450 of first distal shaft branch 112 such that a balloon 892 of first conventional balloon catheter 890 is positioned under branch portion 133 of bifurcated stent 130. Balloon 892 is utilized to deploy bifurcated stent 130 as explained herein. Since there are no balloons mounted on first and second distal shaft branches 112, 118, a combination of conventional balloon catheters having different size balloons may be used to expand respective first and second distal shaft branch 112, 118.

As will be appreciated by those skilled in the art, lumens 450, 456 of first and second distal shaft branches 112, 118, respectively, must be of a sufficient size to accommodate conventional balloon catheters. For example, a conventional balloon catheter typically has a crossing profile of between approximately 1-3 mm (3-9 French units) and thus lumens 450, 456 of first and second distal shaft branches 112, 118, respectively, must be of a slightly larger size in order to insure that the conventional balloon catheter can be advanced through distal shaft branches 112, 118.

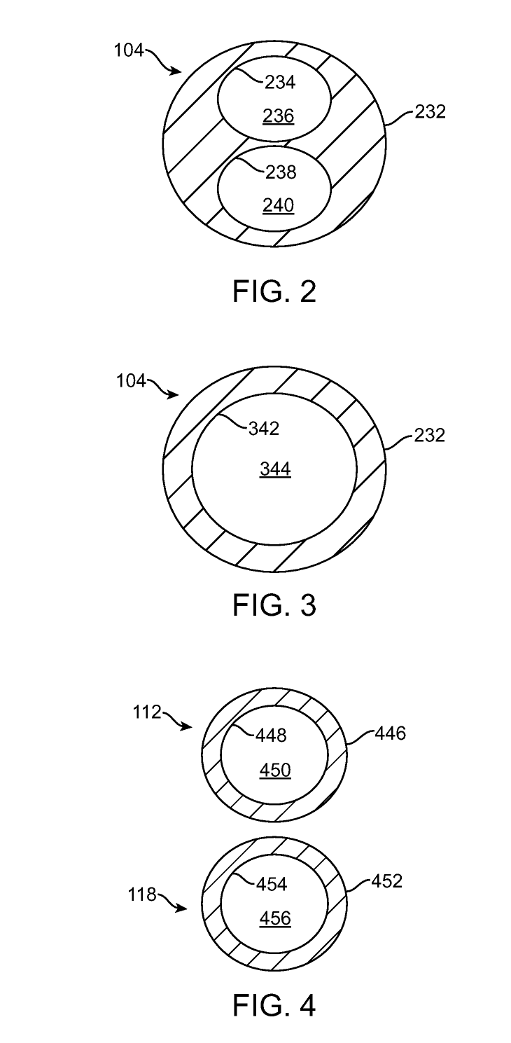

In one embodiment, illustrated in FIG. 2, proximal shaft 104 is biluminal in order to simultaneously accommodate two conventional balloon catheters. FIG. 2 is a cross-sectional view of proximal shaft 104 taken along line A-A of FIG. 1. Proximal shaft 104 has an outside surface 232, and includes first and second lumens 236, 240. First lumen 236 is defined by a first inside surface 234, and second lumen 240 is defined by a second inside surface 238. First catheter port 126 communicates with first lumen 236 that extends through proximal shaft 104 to fluidly communicate with lumen 450 of first distal shaft branch 112 for receiving a first conventional balloon catheter there through. Second catheter port 128 communicates with second lumen 240 that extends through proximal shaft 104 to fluidly communicate with lumen 456 of second distal shaft branch 118 for receiving a second conventional balloon catheter there through. Thus, a first conventional balloon catheter having a balloon of a first size may be inserted through lumens 236, 450 (of proximal shaft 104 and first distal shaft branch 112, respectively) in order to expand or deploy first branch portion 133 of bifurcated stent 130 mounted on first distal shaft branch 112. A second conventional balloon catheter having a balloon of a second size may be inserted through lumens 240, 456 (of proximal shaft 104 and second distal shaft branch 118, respectively) in order to expand or deploy second branch portion 135 of bifurcated stent 130 mounted on second distal shaft branch 118. The sizes of the balloons of the conventional balloon catheters, aforementioned first size and second size, may be the same or may be different. As such, the clinician may custom-select appropriate balloon sizes to best treat the bifurcation. Deployment of first and second branch portions 133, 135 of bifurcated stent 130 may partially deploy trunk 131 of bifurcated stent 130. After first and second branch portions 133, 135 of bifurcated stent 130 are deployed, the first and second conventional balloon catheters may be retracted such that the balloons thereof are located under trunk 131 of bifurcated stent 130. Trunk 131 may be fully deployed by simultaneously or sequentially re-inflating the balloons of the first and second conventional balloon catheters.

Alternatively, trunk 131 may be deployed by a third conventional balloon catheter having a relatively larger balloon mounted thereon inserted through bifurcate stent delivery system 100. More specifically, first and second branch portions 133, 135 of bifurcated stent 130 may be deployed as described above. Deployment of first and second branch portions 133, 135 of bifurcated stent 130 may partially deploy trunk 131 of bifurcated stent 130. After first and second branch portions 133, 135 of bifurcated stent 130 are deployed, the first and second conventional balloon catheters may be retracted and removed from bifurcate stent delivery system 100. A third conventional balloon catheter having a balloon of a third size is inserted through bifurcate stent delivery system 100 and positioned near junction 110. The size of the balloon of the third conventional balloon catheter, aforementioned third size, is preferably larger than the balloons of the first and second conventional balloon catheters, aforementioned first and second sizes. The balloon of the third conventional balloon catheter is inflated to fully deploy trunk 131 of bifurcated stent 130. By expanding the area near junction 110, the proximal portion of trunk 131 is also expanded and causes trunk 131 to fully expand within the body lumen.

As will be appreciated by those skilled in the art, each lumen 236, 240 of proximal shaft 104 is of a sufficient size to accommodate a conventional balloon catheter. For example, a conventional balloon catheter typically has a profile or an outer diameter of approximately 1-3 mm (3-9 French units) and thus the diameters of lumen 236, 240 of proximal shaft 104 must be of a slightly larger size in order to insure that a conventional balloon catheter can be advanced through catheter 102.

In another embodiment, illustrated in FIG. 3, proximal shaft 104 has only a single lumen 344. FIG. 3 is a cross-sectional view of proximal shaft 104 taken along line A-A of FIG. 1. Proximal shaft 104 has outside surface 232, and includes single lumen 344. Single lumen 344 is defined by inside surface 342. Single lumen 344 extends through proximal shaft 104 to fluidly communicate with lumens 450, 456 of distal shaft branches 112, 118, respectively. Single lumen 344 may be of a sufficient size to simultaneously accommodate two conventional balloon catheters inserted through first catheter port 126 and second catheter port 128, respectively. Thus, a first conventional balloon catheter having a balloon of a first size may be inserted through lumens 344, 450 (of proximal shaft 104 and first distal shaft branch 112, respectively) in order to expand or deploy first branch portion 133 of bifurcated stent 130 mounted on first distal shaft branch 112. Likewise, a second conventional balloon catheter having a balloon of a second size may be inserted through lumens 344, 456 (of proximal shaft 104 and second distal shaft branch 118, respectively) in order to, simultaneously or consecutively, expand or deploy second branch portion 135 of bifurcated stent 130 mounted on second distal shaft branch 118. The sizes of the balloons of the conventional balloon catheters, aforementioned first size and second size, may be the same or may be different. As such, the clinician may select appropriate balloon sizes to best treat the bifurcation. After first and second branch portions 133, 135 of bifurcated stent 130 are deployed, trunk 131 of bifurcated stent 130 may be deployed via retraction of the first and second conventional balloon catheters or via a third conventional balloon catheter inserted through bifurcate stent delivery system 100 as described above.

Alternatively, single lumen 344 may be of a sufficient size to accommodate at least one conventional balloon catheter. In such an embodiment, a first conventional balloon catheter having a balloon of a first size is inserted through lumens 344, 450 (of proximal shaft 104 and first distal shaft branch 112, respectively) in order to expand or deploy first branch portion 133 of bifurcated stent 130 mounted on first distal shaft branch 112. The first conventional balloon catheter is retracted and removed, and subsequently a second conventional balloon catheter having a balloon of a second size is inserted through lumens 344, 456 (of proximal shaft 104 and second distal shaft branch 118, respectively) in order to expand or deploy second branch portion 135 of bifurcated stent 130 mounted on second distal shaft branch 118. Trunk 131 of bifurcated stent 130 may be deployed via retraction of the first and second conventional balloon catheters or via a third conventional balloon catheter inserted through bifurcate stent delivery system 100 as described above.

Outer diameters of first and second distal shaft branches 112, 118, respectively, are generally smaller than the outer diameter of proximal shaft 104. For example, the outer diameters of first and second distal shaft branches 112, 118, respectively, are approximately 0.040 inches-0.070 inches, while the outer diameter of proximal shaft 104 is approximately 0.050 inches-0.120 inches. More particularly, when proximal shaft 104 is biluminal, the outer diameter of proximal shaft 104 is approximately 0.090 inches-0.110 inches. When proximal shaft 104 has a single lumen as described above, the outer diameter of proximal shaft 104 may range from approximately 0.050 inches-0.120 inches, depending on whether the single lumen is sized to simultaneously accommodate two conventional balloon catheters or one conventional balloon catheter at a time. The relatively smaller outer diameters for first and second distal shaft branches 112, 118 allow first and second distal shaft branches 112, 118 to be more flexible than proximal shaft 104. Flexibility in first and second distal shaft branches 112, 118 is generally desirable in order to properly track bifurcated stent 130 to the bifurcation lesion.

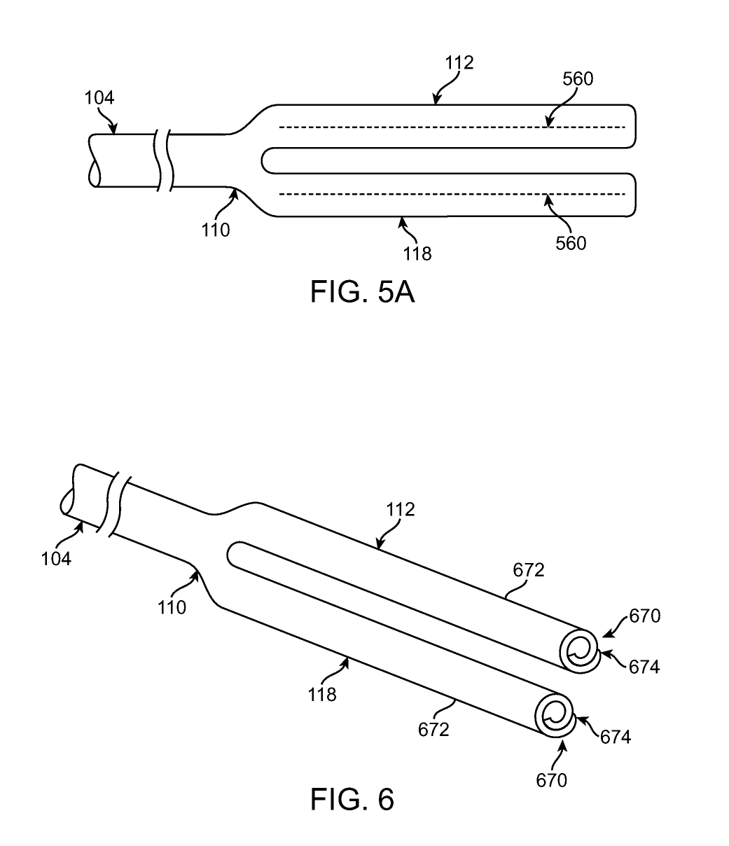

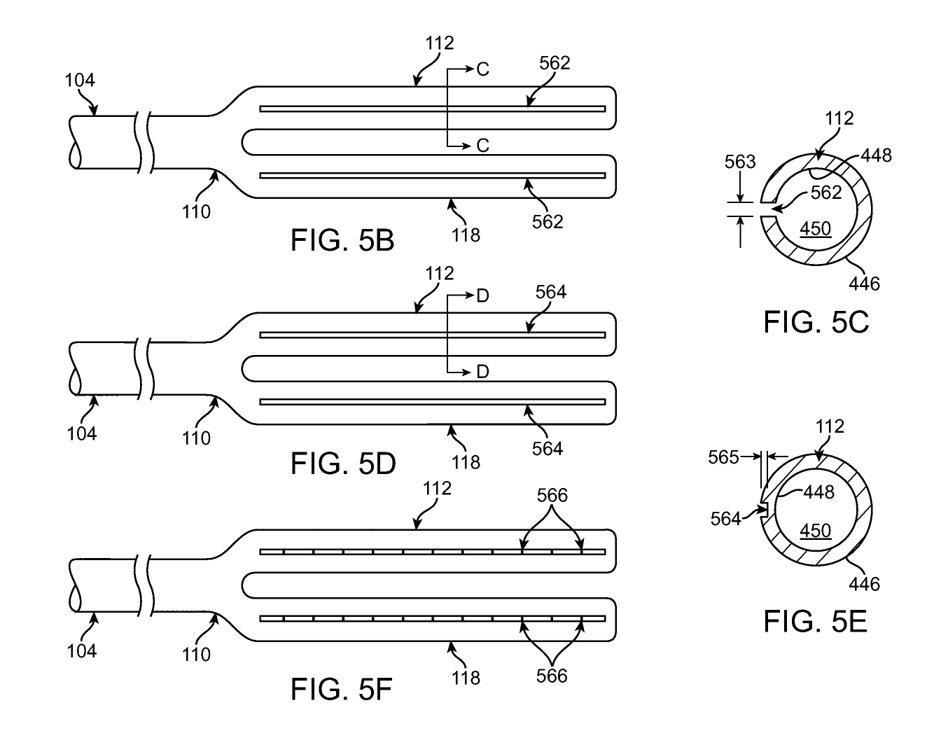

As previously stated, first and second distal shaft branches 112, 118 are constructed to expand in a controlled manner in order to allow bifurcated stent 130 to be expanded or deployed by one or more conventional balloon catheters advanced through catheter 102. In one embodiment, distal shaft branches 112, 118 each include an area of weakness that splits open in response to inflation of a balloon of the conventional balloon catheter. The area of weakness may be of various constructions as illustrated in FIGS. 5A-5F. In FIG. 5A, the area of weakness includes perforations or serrations 560. Perforations or serrations 560 include a series of holes in the form of one or more longitudinal lines provided by perforating first and second distal shaft branches 112, 118. Although a straight line of perforations 560 is shown in FIG. 5A, a wavy or zig-zag pattern of perforations 560 may be utilized without departing from the scope of the present invention.

The area of weakness may also include a slit or slot 562 as illustrated in FIGS. 5B-5C. Slit or slot 562 includes a straight cut, opening, or aperture in the form of one or more longitudinal lines provided by scoring or cutting first and second distal shaft branches 112, 118. As shown in FIG. 5C, slit or slot 562 has a width 563 that may be greater or equal to zero. In other words, slit or slot 562 may include a cut with approximately zero width or may include an opening or aperture with a narrow width 563. Slit or slot 562 has a depth that extends from the inside surface 448 of first distal shaft branch 112 to the outside surface 446 of first distal shaft branch 112. Alternatively, as illustrated in FIGS. 5D-5E, the area of weakness may include a groove 564. Groove 564 has a depth 565 that extends only partially within the material of first distal shaft branch 112, and thus does not extend completely through the outside surface 446 to the inside surface 448 of first distal shaft branch 112.

The area of weakness may also include connectors 566 that are breakable by the inflation of a balloon. For example, connectors 566 may be an adhesive which has a bond strength breakable by the inflation of a balloon. Preferably, the adhesive has a low shear strength and high tensile strength so that the adhesive bond readily disengages upon inflation of a balloon. One example adhesive is NUVA-SIL 5088, which is sold by Loctite Corporation located in Newington, Conn. With this adhesive, a force of approximately 10 to 100 P.S.I. is required to disengage the adhesive. Connectors 566 may also be formed of any polymeric material that is breakable by the inflation of a balloon and may have any structure such as a band or piece of material. Connectors 566 may be used in conjunction with slit or slot 562, as well as groove 564, to ensure that first and second distal shaft branches 112, 118 do not split apart until inflation of a balloon.

In yet another embodiment illustrated in FIG. 6, first and second distal shaft branches 112, 118 are each constructed of a coiled tube 672 that unrolls in response to inflation of a balloon of a conventional balloon catheter in order to allow bifurcated stent 130 to be expanded or deployed by the balloon, herein referred to as an unrolling configuration 670. Coiled tube 672 wraps into a tight spiral configuration having a first diameter for delivery to the treatment site, and unrolls or expands to a second diameter which is larger than the first diameter for deployment of bifurcated stent 130. Unrolling configuration 670 may be accomplished utilizing elastic or shape memory characteristics of a plastic material. More specifically, distal shaft branches 112, 118 may be formed from a polymeric sheet having elastic or shape memory characteristics rolled into coiled tube 672. Inflation of a balloon of a conventional balloon catheter exerts pressure or force onto coiled tube 672 and causes coiled tube 672 to unroll or expand to a second diameter which is larger than the first diameter such that the balloon may further deploy bifurcated stent 130. Upon deflation of the balloon and retraction of the conventional balloon catheter, coiled tube 672 recoils to a size sufficient for bifurcate stent delivery system 100 to be retracted.

In another embodiment, unrolling configuration 670 may be accomplished utilizing an optional adhesive. Distal shaft branches 112, 118 may be formed from a polymeric sheet rolled into coiled tube 672. Adhesive (not shown) having a bond strength breakable by the inflation of a balloon may be applied on the inside surface of an outside end 674 of coiled tube 672 so that outside end 674 is attached to the inside surface of the immediately adjacent layer of coiled tube 672. As described above in relation to FIG. 5F, the adhesive has a low shear strength and high tensile strength so that the adhesive bond readily disengages upon inflation of a balloon. One example adhesive is NUVA-SIL 5088, which is sold by Loctite Corporation located in Newington, Conn. With this adhesive, a force of approximately 10 to 100 P.S.I. is required to disengage the adhesive. The adhesive maintains coiled tube 672 in a coiled or spiral configuration having a first diameter sufficient for delivery to the treatment site. Inflation of a balloon of a conventional balloon catheter breaks the adhesive bond and causes coiled tube 672 to unroll or expand to a second diameter which is larger than the first diameter.

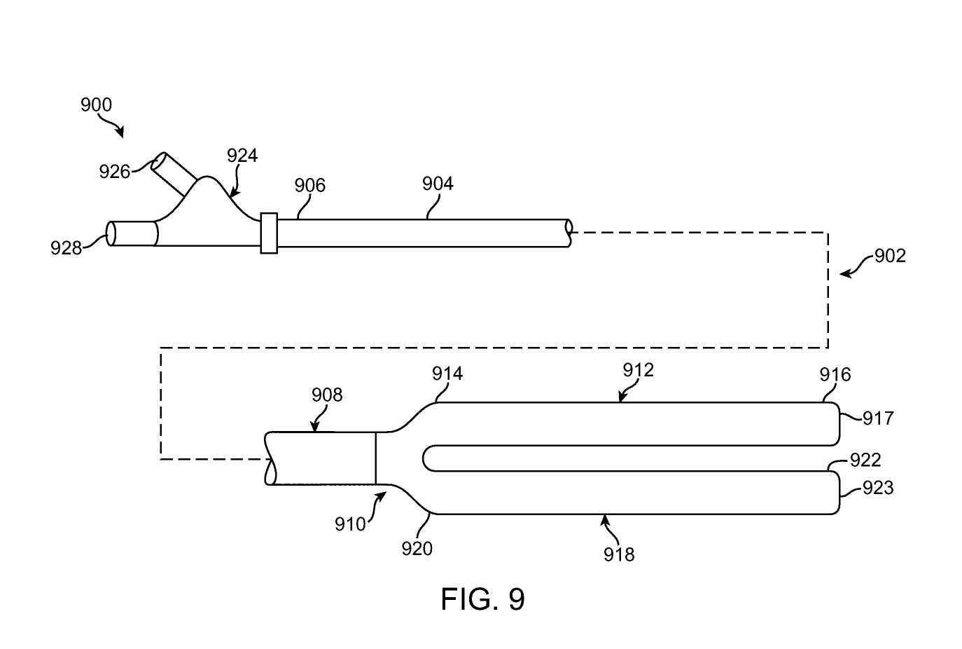

In another embodiment illustrated in FIG. 9, at least a portion of each of the first and second distal shaft branches are constructed out of a material that expands in response to inflation of a balloon of the conventional balloon catheter without including an additional area of weakness as described above. Bifurcate stent delivery system 900 includes catheter 902 for delivering a bifurcated stent (not shown for clarity) mounted on a distal portion thereof. Catheter 902 includes a proximal shaft 904 having a proximal end 906 attached to a hub 924 and a distal end 908 attached to first and second distal shaft branches 912, 918, respectively, via a junction 910. First and second distal shaft branches 912, 918 separately and independently extend from distal end 908 of proximal shaft 904. Hub 924 includes a first catheter port 926 and a second catheter port 928 for accommodating insertion of one or more conventional balloon catheters and/or guidewires within first distal shaft branch 912 and second distal shaft branch 918 of catheter 902. First distal shaft branch 912 includes a proximal end 914 and a distal end 916, wherein distal end 916 includes a guidewire exit port 917. Second distal shaft branch 918 includes a proximal end 920 and a distal end 922, wherein distal end 922 includes a guidewire exit port 923. A bifurcated stent (not shown) may be mounted on first and second distal shaft branches 912, 918 at a location between proximal ends 914, 920 and distal ends 916, 922 of first and second distal shaft branches 912, 918, respectively.

First and second distal shaft branches 912, 918 are each constructed to expand in order to allow the bifurcated stent to be expanded or deployed by one or more conventional balloon catheter advanced there through. At least an integral portion of first and second distal shaft branches 912, 918 is made of a highly elastic polymeric material that is suitable for use in catheters so long as the selected material will elastically expand in response to inflation of a balloon of the conventional balloon catheter there under. For example, silicone rubber and latex rubber could be used. Preferably, the material is an elastomeric material such as NUSIL silicone grade Med10-6640, which has a very high elongation before breakage. When a balloon of a conventional balloon catheter is inflated, the material of first and second distal shaft branches 912, 918 is expanded to the extent that it allows the bifurcated stent mounted thereon to be expanded or deployed without exceeding its elastic limit.

In the embodiment depicted in FIG. 9, junction 910 and the entire first and second distal shaft branches 912, 918 are constructed out of the highly elastic polymeric material and attached or connected to the distal end 908 of catheter 902. Junction 910 and first and second distal shaft branches 912, 918 have a substantially U-shaped configuration, and may be formed via extrusion resulting in one continuous structure. Proximal shaft 904 and the U-shaped structure may then be welded, fused, bonded, or otherwise joined together. In another embodiment, only first and second distal shaft branches 912, 918 are constructed out of the highly elastic polymeric material. In yet another embodiment, only a portion of first and second distal shaft branches 912, 918 are constructed out of the highly elastic polymeric material.

In order to deploy bifurcated stent 130, bifurcate stent delivery system 100 must be tracked to and properly positioned at the bifurcation lesion. In general, two guidewires (not shown) are introduced into the main vessel, one guidewire extending through the bifurcation into one branch vessel, and the other guidewire extending through the bifurcation into the other branch vessel. Bifurcate stent delivery system 100 is then tracked over the two guidewires such that bifurcated stent 130 in positioned at the bifurcation lesion. A first guidewire extends in lumen 450 of first distal shaft branch 112 and a second guidewire extends in lumen 456 of second distal shaft branch 118 until junction 110. At junction 110, if proximal shaft 104 includes a single lumen 344 of a sufficient size to simultaneously accommodate two conventional balloon catheters inserted through first catheter port 126 and second catheter port 128, respectively, both guidewires extend within single lumen 344 of proximal shaft 104. If proximal shaft 104 includes dual lumens 236, 240, a first guidewire extends in lumen 236 of proximal shaft 104 and a second guidewire extends in lumen 240 of proximal shaft 104 until junction 110. Any known method may be utilized for initially introducing two guidewires into the main vessel, one guidewire extending through the bifurcation into one branch vessel, and the other guidewire extending through the bifurcation into the other branch vessel. In addition, it is not necessary that both guidewires be in place prior to advancing bifurcate stent delivery system 100. For example, a first guidewire may be inserted into the vessel and bifurcate stent delivery system 100 may be tracked to the bifurcation lesion over only the first guidewire. Subsequently, a second guidewire may be inserted into bifurcate stent delivery system 100. Any appropriate method may be used for tracking and placing bifurcate stent delivery system 100 at the bifurcation lesion.

Once bifurcate stent delivery system 100 having the two guidewires extending there through is in place at the bifurcation lesion as desired, the guidewires may be removed and conventional balloon catheters are inserted through catheter ports 126, 128 and tracked through distal shaft branches 112, 118 to a position in which a balloon of the conventional balloon catheters is placed under at least a portion of bifurcated stent 130. With the guidewires removed, bifurcate stent delivery catheter 100 acts as a guide catheter for tracking the conventional balloon catheters to the site of bifurcated stent 130. The ability of bifurcate stent delivery catheter 100 to act as a guide catheter is especially applicable in the embodiment in which proximal shaft 104 includes dual lumens 236, 240. First and second conventional balloon catheters inserted through first catheter port 126 and second catheter port 128, respectively, are individually tracked through dual lumens 236, 240. Dual lumens 236, 240 ensure that the first and second conventional balloon catheters do not interfere with each other as they are being tracked to bifurcated stent 130.

Alternatively, one or both guidewires may be left in place within bifurcate stent delivery system 100 and conventional balloon catheters inserted through catheter ports 126, 128 will be tracked over the guidewires. It may be desirable to leave one or both guidewires in place in the embodiment in which proximal shaft 104 includes a single lumen 344 of a sufficient size to simultaneously accommodate two conventional balloon catheters. The guidewires may assist in properly positioning first and second conventional balloon catheters within distal shaft branches 112, 118.

Regardless of whether the guidewires are removed or left in place, the balloons of the conventional balloon catheters are advanced until they are in position under at least a portion of bifurcated stent 130 and then are inflated either simultaneously or sequentially. In response to the inflation of the balloon inserted therein, each distal shaft branch 112, 118 splits open or otherwise expands in a controlled manner permitting the balloon to expand and thus deploy or expand a portion of bifurcated stent 130. Portions of bifurcated stent 130, i.e., trunk 131 and side branch portions 133, 135, may be deployed simultaneously or sequentially as desired by the clinician.

Once distal shaft branches 112, 118 split or expand in response to the inflation of a balloon of a conventional balloon catheter, bifurcated stent 130 is deployed or expanded within a body lumen. Upon deflation of the balloon catheter, first and second distal shaft branches 112, 118 resume a small enough diameter to be retracted from the body lumen such that catheter 102 may be removed from the patient. First and second distal shaft branches 112, 118 are thus preferably formed of a material having elastic or shape memory characteristics such that first and second distal shaft branches 112, 118 recoil upon deflation of the balloon catheter to a size sufficient for bifurcate stent delivery system 100 to be retracted. In addition, an optional sheath (not shown) may be utilized to assist in minimizing first and second distal shaft branches 112, 118 such that they resume a small enough diameter to be retracted from the body lumen such that catheter 102 may be removed from the patient.

Proximal shaft 104 may be formed of any appropriate polymeric material. Non-exhaustive examples of material for proximal shaft 104 are polyethylene terephalate (PET), which allows for very thin walls while withstanding high pressures; nylon, which provides a soft material; and polyethylene, which is advantageous for its compatibility with new angioplasty techniques, such as lasers; PEBAX; or combinations of any of these, either blended or co-extruded. Optionally, proximate shaft 104 or some portion thereof may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. For example, at least proximal end 106 of proximal shaft 104 may in some instances be formed from a reinforced polymeric tube. As a further alternative, at least proximal end 106 of proximal shaft 104 may in some instances be formed from a metal, highly elastic, or super elastic hypotube material.

As previously mentioned, flexibility in the distal shaft branches 112, 118 is generally desirable in order to properly track bifurcated stent 130 to the bifurcation lesion. Thus, a generally more flexible polymeric material such as PEBAX is a preferred material for distal shaft branches 112, 118. However, distal shaft branches 112, 118 may be formed of any suitable flexible polymeric material, including polyethylene terephalate (PET), nylon, polyethylene, PEBAX, or combinations of any of these, either blended or co-extruded. In another embodiment as described above, distal shaft branches 112, 118 may be constructed out of an elastomeric material that elastically expands in response to inflation of a balloon of the conventional balloon catheter without including an area of weakness. For example, silicone rubber, latex rubber, or NUSIL silicone grade Med10-6640 which has a very high elongation before break, may be used.

Bifurcate stent delivery system 100 has a relatively simple structure of three tubular components (i.e., proximal shaft 104, first distal shaft branch 112, and second distal shaft branch 118) and thus is easy to manufacture in comparison to other stent delivery systems that include a balloon or other deployment mechanism for a stent loaded thereon. Junction 110 is a transition area between proximal shaft 104 and distal shaft branches 112, 118. Distal end 108 of proximal shaft 104 is attached to distal shaft branches 112, 118 such that distal shaft branches 112, 118 extend separately from distal end 108 of proximal shaft 104. Proximal shaft 104 and distal shaft branches 112, 118 may be separate tubular components that are welded, fused, bonded, or otherwise joined together. However, if the same material is used for proximal shaft 104 and distal shaft branches 112, 118, then catheter 102 may be formed via extrusion resulting in one continuous structure. If proximal shaft 104 includes single lumen 344, junction 110 serves as a transition area for splitting single lumen 344 into communication with each of lumens 450, 456 of distal shaft branches 112, 118. If proximal shaft 104 includes dual lumens 236, 240, junction 110 serves as a transition area for transitioning first lumen 236 into communication with lumen 450 of first distal shaft branch 112 and for transitioning second lumen 240 into communication with lumen 456 of second distal shaft branch 118.

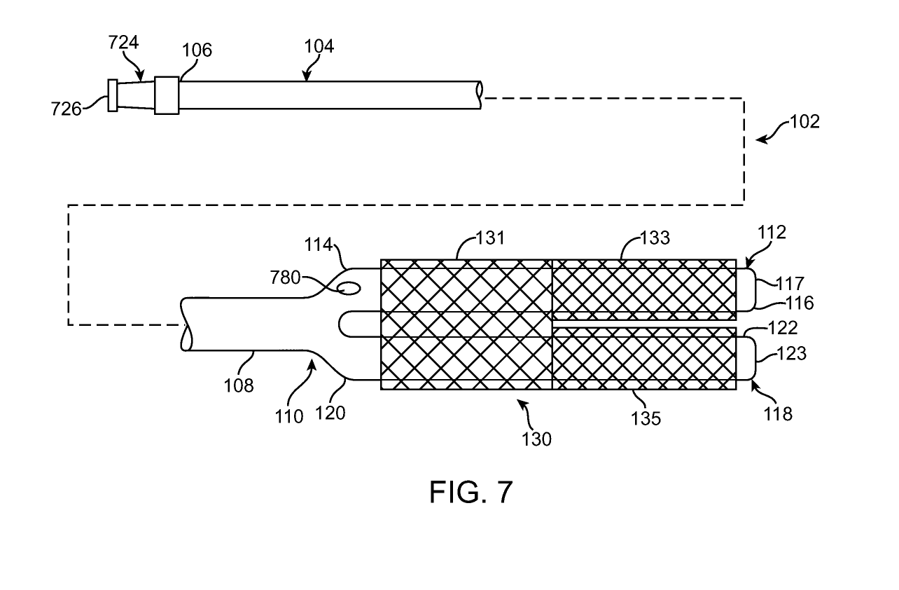

It is to be understood by those skilled in the art that bifurcate stent delivery system 100 of the present invention, described in detail above, may be modified to function as a hybrid over-the-wire and rapid-exchange (RX) catheter. A RX catheter, as opposed to an OTW catheter, has a guidewire shaft that extends within only the distalmost portion of the catheter. Thus, during a PTCA or stent delivery procedure only the distalmost portion of a RX catheter is tracked over a guidewire. FIG. 7 illustrates a hybrid over-the-wire and RX catheter in which first distal shaft branch 112 is modified to include rapid-exchange functionality. In such a hybrid over-the-wire and RX catheter, proximal shaft 104 is required to have at least one lumen extending there through, wherein the lumen is of a sufficient size to accommodate a conventional balloon catheter. Hub 724 includes a catheter port 726 to accommodate insertion of a conventional balloon catheter. A guidewire port 780 is located at a proximal end 114 of distal shaft branch 112. Guidewire port 780 communicates with lumen 450 of first distal shaft branch 112 for receiving a guidewire there through. Guidewire port 780 is of a sufficient size to allow entry of a conventional balloon catheter. Guidewire port 780 and a first guidewire received there through may be used to track and properly position bifurcate stent delivery system 100 at the bifurcation lesion. A first conventional balloon catheter would be tracked over the first guidewire for deploying branch portion 133 of bifurcated stent 130 located on first distal shaft branch 112. Second distal shaft branch 118 functions in an over-the-wire configuration for receiving a second guidewire and/or second conventional balloon catheter there through as described above in relation to FIGS. 1-3. The second conventional balloon catheter is inserted through catheter port 726 and tracked through distal shaft branches 118 to a position in which a balloon of the second conventional balloon catheters is placed under branch portion 135 of bifurcated stent 130.

To accomplish a hybrid over-the-wire and RX catheter, one skilled in the art can appreciate that one of either first distal shaft branch 112 or second distal shaft branch 118 is modified to include a guidewire port for receiving a guidewire and a conventional balloon catheter tracked there over. Thus, rather than modifying first distal shaft branch 112 to include a guidewire port 780 as described in relation to FIG. 7, second distal shaft branch 118 may be modified to include rapid-exchange functionality while first distal shaft branch 112 functions in an over-the-wire configuration. In addition, as will be apparent to those skilled in the art, both first and second distal shaft branches 112, 118 may be modified to function as a dual rapid exchange (RX) catheter by including guidewire ports for receiving a guidewire and a conventional balloon catheter tracked there over in each of first and second distal shaft branches 112, 118.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A stent delivery system for delivering a stent to a bifurcation, comprising:
a proximal shaft having a proximal end and a distal end with a lumen extending there through;
a first distal shaft branch having a first branch lumen extending there through with an area of weakness along a length thereof; and
a second distal shaft branch having a second branch lumen extending there through with an area of weakness along a length thereof,
wherein the first and second distal shaft branches separately extend from the distal end of the proximal shaft such that the first and second branch lumens are in fluid communication with the proximal shaft lumen, and
wherein each of the areas of weakness along the first and second distal shaft branches splits open to an expanded configuration when a balloon of a balloon catheter is inflated within its respective lumen.

2. The stent delivery system of claim 1, further comprising:
a first stent mounted over the area of weakness on the first distal shaft branch and a second stent mounted over the area of weakness on the second distal shaft branch.

3. The stent delivery system of claim 1, further comprising:
a bifurcated stent having a first leg mounted over the area of weakness on the first distal shaft branch, a second leg mounted over the area of weakness on the second distal shaft branch, and a trunk mounted over the area of weakness on the first distal shaft branch and the area of weakness on the second distal shaft branch.

4. The stent delivery system of claim 3, wherein at least the first leg of the bifurcated stent is expanded when the area of weakness of the first distal shaft branch is in the expanded configuration.

5. The stent delivery system of claim 3, wherein at least the second leg of the bifurcated stent is expanded when the area of weakness of the second distal shaft branch is in the expanded configuration.

6. The stent delivery system of claim 3, wherein the trunk of the bifurcated stent is expanded when the area of weakness of the second distal shaft branch and the area of weakness of the first distal shaft branch are both in the expanded configurations.

7. The bifurcate stent delivery system of claim 1, wherein each of the areas of weakness is a line of perforations.

8. The bifurcate stent delivery system of claim 1, wherein each of the areas of weakness is selected from a group consisting of a slit, a slot, and a groove.

9. The bifurcate stent delivery system of claim 1, wherein each of the areas of weakness includes a connector breakable in response to inflation of a balloon of the balloon catheter.

10. The bifurcate stent delivery system of claim 1, wherein the proximal shaft has a first lumen extending there through and a second lumen extending there through, the first lumen of the proximal shaft in fluid communication with the first branch lumen of the first distal shaft branch and the second lumen of the proximal shaft in fluid communication with the second branch lumen of the second distal shaft branch, and wherein the first lumen and the second lumen of the proximal shaft are each of a sufficient size such that a balloon catheter may be advanced there through.

11. The bifurcate stent delivery system of claim 1, wherein the proximal shaft has only a single lumen extending there through, the single lumen of the proximal shaft in fluid communication with both the first and second branch lumens, the single lumen being of a sufficient size such that at least one balloon catheter may be advanced there through.

12. The bifurcate stent delivery system of claim 11, wherein the single lumen is of a sufficient size such that two balloon catheters may simultaneously be advanced there through.

13. A stent delivery system for delivering a stent to a bifurcation, comprising:
a proximal shaft having a proximal end and a distal end with a lumen extending there through;
a first distal shaft branch formed of a coiled tube having a first branch lumen extending there through; and
a second distal shaft branch formed of a coiled tube having a second branch lumen extending there through,
wherein the first and second distal shaft branches separately extend from the distal end of the proximal shaft such that the first and second branch lumens are in fluid communication with the proximal shaft lumen, and
wherein each of the first and second distal shaft branches unrolls to an expanded configuration when a balloon of a balloon catheter is inflated within its respective lumen.

14. The stent delivery system of claim 13, further comprising:
a first stent mounted on the first distal shaft branch and a second stent mounted on the second distal shaft branch.

15. The stent delivery system of claim 13, further comprising:
a bifurcated stent having a first leg mounted on the first distal shaft branch, a second leg mounted on the second distal shaft branch, and a trunk mounted over the area of weakness on the first distal shaft branch and the area of weakness on the second distal shaft branch.

16. The stent delivery system of claim 15, wherein at least the first leg of the bifurcated stent is expanded when the coiled tube of the first distal shaft branch is in the expanded configuration.

17. The stent delivery system of claim 15, wherein at least the second leg of the bifurcated stent is expanded when the coiled tube of the second distal shaft branch is in the expanded configuration.

18. The stent delivery system of claim 15, wherein the trunk of the bifurcated stent is expanded when the area of weakness of the second distal shaft branch and the area of weakness of the first distal shaft branch are both in the expanded configurations.

19. The bifurcate stent delivery system of claim 13, wherein the proximal shaft has a first lumen extending there through and a second lumen extending there through, the first lumen of the proximal shaft in fluid communication with the first branch lumen and the second lumen of the proximal shaft in fluid communication with the second branch lumen, and wherein each of the first lumen and the second lumen of the proximal shaft are of a sufficient size such that a balloon catheter may be advanced there through.

20. The bifurcate stent delivery system of claim 13, wherein the proximal shaft has only a single lumen extending there through, the single lumen of the proximal shaft in fluid communication with both the first and second branch lumens, the single lumen being of a sufficient size such that a balloon catheter may be advanced there through.

21. The bifurcate stent delivery system of claim 20, wherein the single lumen is of a sufficient size such that two balloon catheters may simultaneously be advanced there through.

22. A stent delivery system for delivering a stent to a bifurcation, comprising:
a proximal shaft having a proximal end and a distal end with a lumen extending there through;
a first distal shaft branch having a first branch lumen extending there through; and
a second distal shaft branch having a second branch lumen extending there through,
wherein the first and second distal shaft branches separately extend from the distal end of the proximal shaft such that the first and second branch lumens are in fluid communication with the proximal shaft lumen, and
wherein at least an integral portion of each of the first and second distal shaft branches is formed of a polymeric material that expands without exceeding its elastic limit to an expanded configuration when a balloon of a balloon catheter is inflated within its respective lumen.

23. The stent delivery system of claim 22, further comprising:
a first stent mounted over the first distal shaft branch and a second stent mounted over the second distal shaft branch.

24. The stent delivery system of claim 22, further comprising:
a bifurcated stent having a first leg mounted over the first distal shaft branch, a second leg mounted over the second distal shaft branch, and a trunk mounted over the first distal shaft branch and the second distal shaft branch.

25. A method of treating a bifurcation by expanding or deploying a bifurcated stent, the method comprising the steps:
tracking a bifurcate stent delivery system to a bifurcation lesion, wherein the bifurcate stent delivery system includes a proximal shaft having a proximal end and a distal end, a first distal shaft branch having a lumen extending there through and a second distal shaft branch having a lumen extending there through, the first and second distal shaft branches separately extending from the distal end of the proximal shaft, wherein the lumens of the first and second distal shaft branches each are of a sufficient size such that a balloon catheter may be advanced there through, and a bifurcated stent attached to the first and second distal shaft branches;
tracking a first balloon catheter through the bifurcate stent delivery system such that a balloon of the first balloon catheter is located under at least a portion of the bifurcated stent;
tracking a second balloon catheter through the bifurcate stent delivery system such that a balloon of the second balloon catheter is located under at least a portion of the bifurcated stent;
inflating the balloon of the first balloon catheter, wherein the first distal shaft branch includes an area of weakness that splits open in response to inflation of the balloon of the first balloon catheter in order to allow a portion of the bifurcated stent to be expanded or deployed;
inflating the balloon of the second balloon catheter, wherein the second distal shaft branch includes an area of weakness that splits open in response to inflation of the balloon of the second balloon catheter in order to allow a portion of the bifurcated stent to be expanded or deployed; and
expanding or deploying the bifurcated stent in the bifurcation lesion.
